(19) United States
(12) Patent Application Publication
(10) Pub. No.: US 2026/0167600 A1
(43) Pub. Date: Jun. 18, 2026

(54) PARA-PHENYLENEDIAMINE DERIVATIVES FROM KETONES AND ALDEHYDES FOR ANTIOZONANT AND ANTIOXIDANT APPLICATIONS

(71) Applicant: The Goodyear Tire & Rubber Company, Akron, OH (US)

(72) Inventors: Gyusaang Youn, Copley, OH (US); Aaron Patrick Murray, Chardon, OH (US); Margaret Flook Vielhaber, Kent, OH (US); Michael Lester Kerns, Medina, OH (US)

(21) Appl. No.: 18/985,905

(22) Filed: Dec. 18, 2024

(51) Int. Cl. C07C 209/02; B60C 1/00; C08K 5/18

(52) U.S. Cl. CPC C07C 209/02; B60C 1/00; C08K 5/18

(57) ABSTRACT

In one aspect, the disclosure relates to new antioxidant and/or antiozonant compounds and methods of making the same. The compounds have migration profiles and performance in rubber compositions that are comparable to those of known antiozonants such as 6PPD. Also disclosed herein are rubber compositions including the disclosed compounds and tires and/or other rubber articles including the rubber compositions.

### BACKGROUND

Rubber articles such as vehicle tires are degraded by atmospheric oxygen and ozone, leading to discoloration as well as structural defects such as cracking while in a strained state such as during road use. Ozone can also react with some polymers in tires in a way that generates free radicals. Damage caused by ozone and free radicals can lead to degraded appearance as well as causing performance and safety issues. Thus, antioxidants and antiozonants have been introduced into tire sidewalls to protect against the effects of atmospheric oxygen and ozone.

6PPD is a compound having antiozonant properties that is frequently incorporated into rubber articles including vehicle tires.

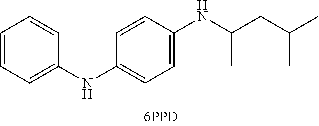 [SMILES: CC(C)CC(C)Nc1ccc(Nc2ccccc2)cc1]

This compound migrates over time to the surface of the vehicle tires, essentially forming a film that scavenges ozone before the ozone can degrade the rubber. 6PPD essentially replenishes itself at the tire surface through continuous blooming. Due to its high reactivity, however, 6PPD may transform into byproducts of 6PPD. For example, 6PPD-quinone is an oxidized byproduct of 6PPD in tires.

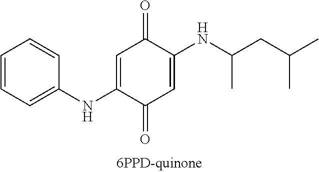 [SMILES: CC(C)CC(C)NC1=CC(=O)C(Nc2ccccc2)=CC1=O]

Thus, a replacement compound having comparable or better antioxidant, antiozonant, and migration properties through rubber is desired.

### SUMMARY

In accordance with the purpose(s) of the present disclosure, as embodied and broadly described herein, the disclosure, in one aspect, relates to new antioxidant and/or antiozonant compounds and methods of making the same. The compounds have migration profiles and performance in rubber compositions that are comparable to those of known antiozonants such as 6PPD. In one aspect, however, the reaction products of the disclosed compounds with ozone are non-toxic. Also disclosed herein are rubber compositions including the disclosed compounds and tires and/or other rubber articles including the rubber compositions.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a solvent,” “a base,” or “an antiozonant property,” include, but are not limited to, mixtures or combinations of two or more such solvents, bases, or antiozonant properties, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g, the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, the term “prevent” or “preventing” refers to precluding, averting, obviating, forestalling, stopping, or hindering something from happening, especially by advance action. The term “prevent” or “preventing” also includes reducing the severity of a particular issue or problem. For example, the compounds described herein can reduce the severity of cracking of a rubber polymer or a rubber composition.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

“Substituted” as used herein refers to replacement of a carbon atom with a heteroatom such as N, S, P, or the like (e.g., conversion of aryl to heteroaryl such as replacing a carbon in benzene with nitrogen to form pyridine) or replacement of a hydrogen atom with another atom or group including, but not limited to, an alkyl group, alkenyl group, alkynyl group, halogen (e.g. F, Cl, Br, I), aryl group, heteroaryl group, or the like. It is understood when substitutions are incorporated into a chemical group or molecule that all standard valence rules will be satisfied.

Unless otherwise specified, pressures referred to herein are based on atmospheric pressure (i.e. one atmosphere).

### Antioxidant/Antiozonant Compounds

In one aspect, the compounds and salts disclosed herein have Formula I:

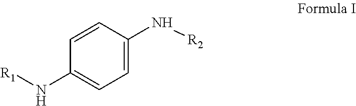

wherein R₁ and R₂ independently comprise linear or branched C3-C20 alkyl; substituted or unsubstituted C3-C20 cycloalkyl, linear or branched C3-C20 alkenyl; fused or bridged C3-C20 cycloalkyl, substituted or unsubstituted C₅-C₁₀ heteroaryl, or substituted or unsubstituted C₅-C₁₀ aryl;
provided that R₂ is not

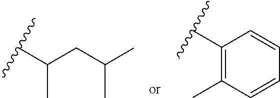

when R₁ is phenyl.

In an aspect, R₁ can be unsubstituted C₅-C₆ aryl or C₅-C₆ aryl substituted with a C₁-C₄ linear or branched alkyl group or a C₅-C₁₀ aryl group. In another aspect, R₁ and R₂ are different from one another; that is, the compound is asymmetric. Further in this aspect, R₁ and R₂ can both be aryl, or R₁ can be aryl while R₂ is alkyl or cycloalkyl, or one of R₁ and R₂ can be alkyl while the other can be cycloalkyl. In some aspects, R₁ can be a phenyl group, and the compound of Formula I can have the structure

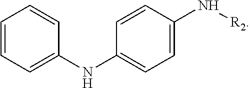

In any of these aspects, the compound can be provided as a salt having a counter ion selected from cations having a +1 or +2 charge such as, for example, Na⁺, K⁺, Ca²⁺, Mg²⁺, Zn²⁺, or any combination thereof.

In one aspect, the alkenyl substituent can be any linear or branched C3-C10 alkenyl group having from 1-3 lower alkyl substituents on either side of one double bond. In another aspect, fused or bridged cycloalkyl substituent can be selected from adamantyl, norbornyl, decalyl, or any combination thereof.

In another aspect, R₂ can be a C4-C20 alkyl group. In another aspect, R₂ can be a substituted or unsubstituted C3-C₂₀ cycloalkyl group. In still another aspect, R₂ can be a linear or branched C3-C10 alkenyl group having from 1-3 lower alkyl substituents on one double bond. In still another aspect, R₂ is a fused or bridged cycloalkyl group selected from adamantyl, norbornyl, or decalyl. Exemplary R₁ and R₂ groups include, but are not limited to, the following:

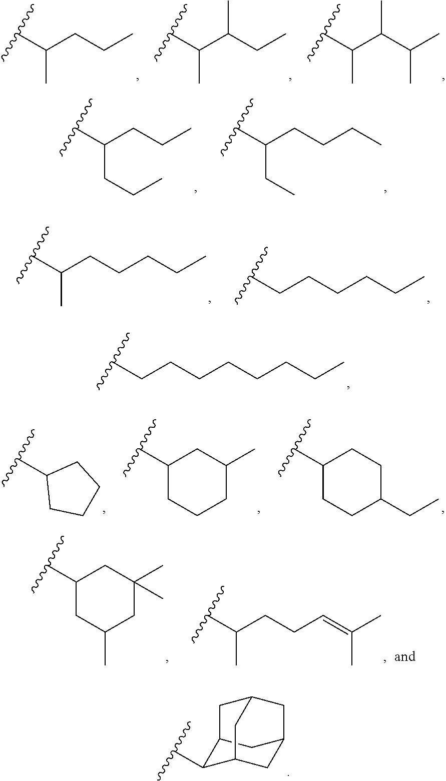

In one aspect, a blend of two or more compounds of Formula I can be applied for improved ozone protection and migrate better through rubber articles made therefrom. In any of these aspects, the compound can be provided as a salt having a counter ion selected from cations having a +1 or +2 charge such as, for example, Na+, K+, Ca2+, Mg2+, Zn2+, or any combination thereof.

Exemplary compounds according to the present disclosure include, but are not limited to, at least the following:

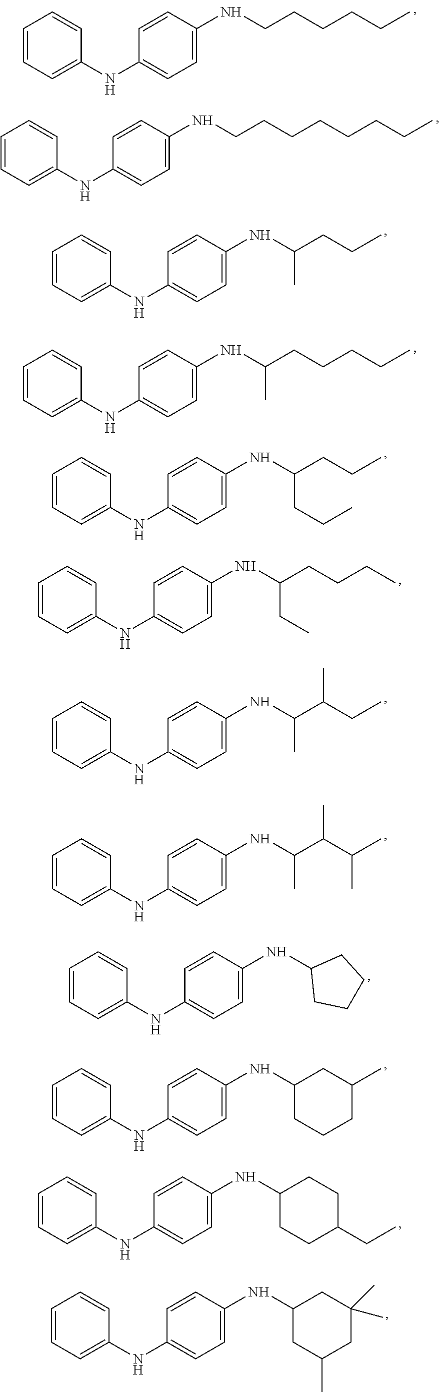

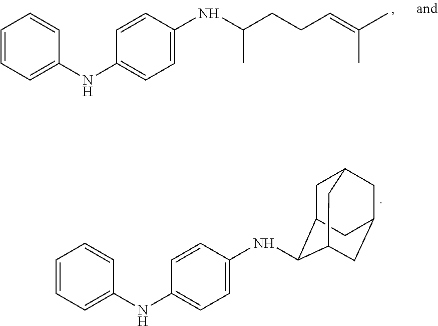 [SMILES: CC(C)=CCCC(C)Nc1ccc(Nc2ccccc2)cc1 ; c1ccc(Nc2ccc(NC3C4CC5CC(C4)CC3C5)cc2)cc1]

In one aspect, the disclosed compounds and their degradation products and reaction products with ozone are non-toxic.

In some aspects, the following compounds are excluded from the present disclosure:

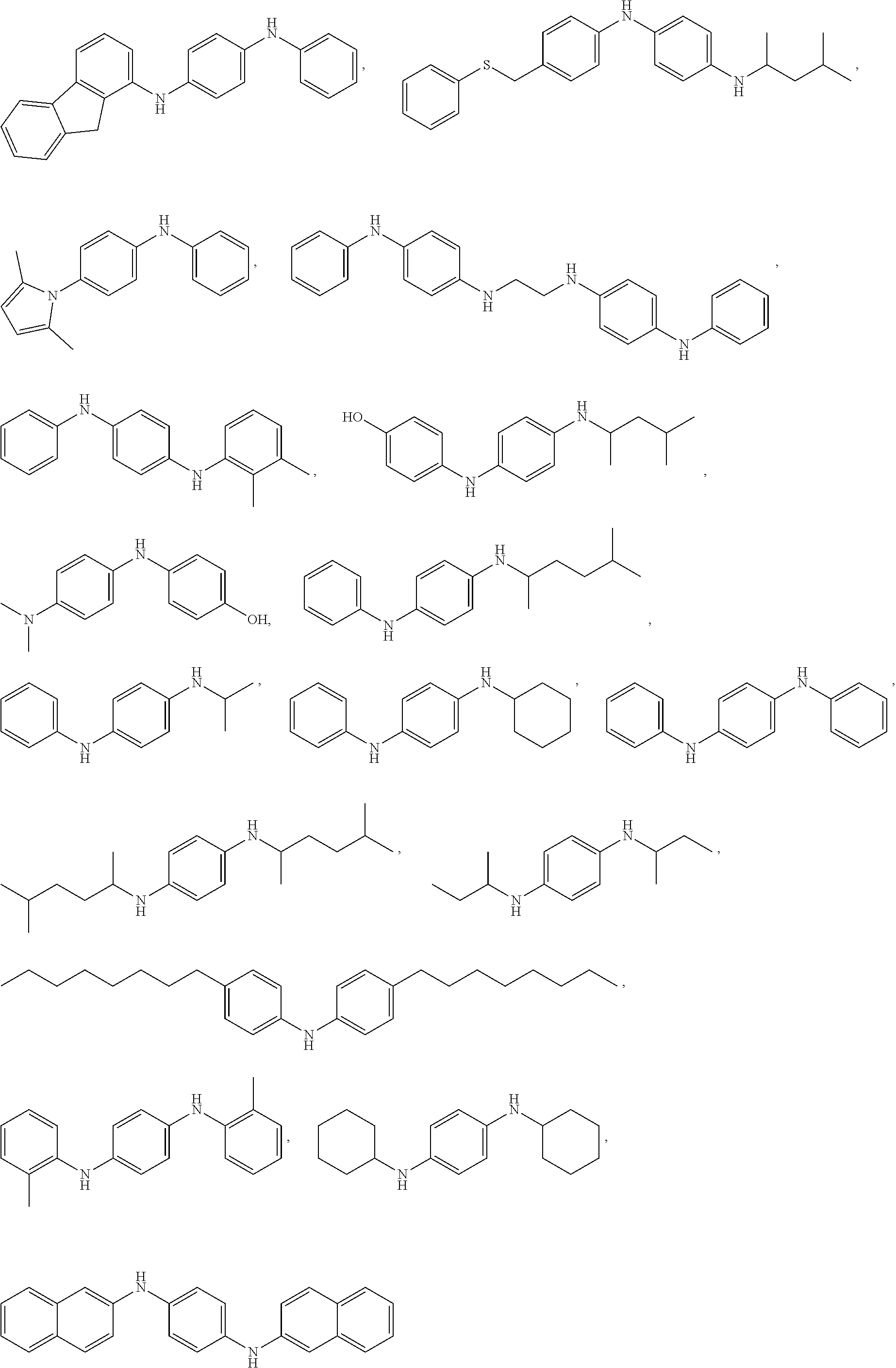

### Methods for Making the Antioxidant/Antiozonant Compounds and Salts

In one aspect, disclosed is a method for making a disclosed compound or salt, the method including admixing a compound of Formula II

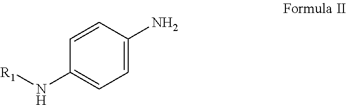

with a carbonyl compound comprising an aldehyde or ketone having Formula III and a reducing agent

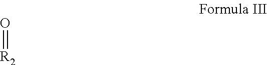

wherein R₁ and R₂ independently comprise linear or branched C3-C20 alkyl; substituted or unsubstituted C3-C20 cycloalkyl, linear or branched C3-C20 alkenyl; fused or bridged C3-C20 cycloalkyl, substituted or unsubstituted C₅-C₁₀ heteroaryl, or substituted or unsubstituted C₅-C₁₀ aryl;
provided that R₂ is not

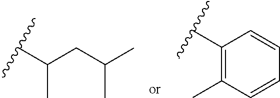

when R₁ is phenyl.

In an aspect, the compound of Formula I is admixed with the carbonyl compound to produce a first composition, then the first composition is admixed with the reducing agent. In some aspects, this step is performed in the presence of acetic acid.

In another aspect, the method further includes treating the compound with a base such as, for example, NaOH, KOH, Ca(OH)₂, Mg(OH)₂, Zn(OH)₂, CaO, K₂CO₃, Na₂CO₃, Na(0), or any combination thereof, to form the salt.

In an aspect, the reducing agent can be NaBH(OAc)₃, NaBH₄, Na(CN)BH₃, or any combination thereof. In one aspect the method can be conducted in a polar aprotic solvent including, but not limited to, tetrahydrofuran (THF), toluene, xylene, or any combination thereof.

In another aspect, disclosed is a second method for making a disclosed compound or salt, the second method including admixing hydroquinone with R₁—NH₂, R₂—NH₂, and a Lewis acid such as, for example, FeCl₃, in a solvent. In an aspect the method can be conducted in a polar aprotic solvent including, but not limited to, tetrahydrofuran (THF), toluene, xylene, or any combination thereof.

In still another aspect, disclosed is a third method for making a disclosed compound or salt, the third method including at least the steps of:
(a) admixing a compound having a structure of Formula II

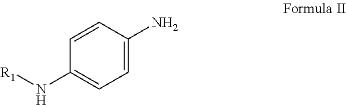

with an aldehyde or ketone having Formula III

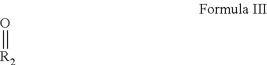

in a solvent to form an intermediate; and
(b) performing catalytic hydrogenation on the intermediate to produce the compound or salt;
wherein R₁ and R₂ independently comprise linear or branched C3-C20 alkyl; substituted or unsubstituted C3-C20 cycloalkyl, linear or branched C3-C10 alkenyl; fused or bridged C3-C20 cycloalkyl, or substituted or unsubstituted C₅-C₁₀ aryl;
provided that R₂ is not

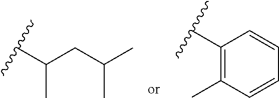

when R₁ is phenyl.

In an aspect the method can be conducted in a polar aprotic solvent including, but not limited to, tetrahydrofuran (THF), toluene, xylene, or any combination thereof. In an aspect, the catalyst for hydrogenation can be selected from using platinum dioxide, cobalt boride, Crabtree's catalyst, HRh[P(C₆H₅)₃]₄, Lindlar catalyst, nickel boride, palladium black, palladium on carbon, Raney nickel, Rh—Pt oxide, Urushibara nickel, Wilkinson's catalyst, or any combination thereof.

### Rubber Compositions and Applications Thereof

The compounds described herein can be incorporated into rubber compositions including one or more rubber polymers. In another aspect, in the rubber compositions, the disclosed compounds have at least one antiozonant property, at least one antiozonant property, or any combination thereof. In a further aspect, the antiozonant property, the antioxidant property, or both can be selected from prevention of cracking of the one or more rubber polymers, slowing of cracking of the one or more rubber polymers, prevention of a color change of the one or more rubber polymers, preservation of flexibility of the one or more rubber polymers, or any combination thereof.

In an aspect, the one or more rubber polymers can be selected from natural rubber, polybutadiene, styrene-butadiene rubber, nitrile rubber, polyisoprene, chloroprene, ethylene-butadiene, hydrogenated butadiene, hydrogenated styrene-butadiene, or any combination thereof. In some aspects, the disclosed rubber compositions include a solution polymerized styrene-butadiene rubber (SBR). In one aspect, the solution polymerization prepared SBR (sSBR) can have a bound styrene content in the range of from about 5 percent to about 50 percent, or from about 9 percent to about 26 percent. In a further aspect, the sSBR can be conveniently prepared by organolithium catalysis in the presence of a hydrocarbon solvent.

In one aspect, a reference to glass transition temperature, or T_{g}, of an elastomer or elastomer composition, when used herein, represents the glass transition temperature(s) of the respective elastomer or elastomer composition in its uncured state or, in the case of an elastomer composition, in some aspects, T_{g} can be measured in a cured state. In an aspect, T_{g} can be suitably determined as a peak midpoint by a differential scanning calorimeter (DSC) using a test standard such as, for example, ASTM D7426 or equivalent.

In one embodiment, the solution polymerized styrene-butadiene rubber has a glass transition temperature in a range of from about −85° C. to about 0° C., or from about −85° C. to about −50° C., or from about −40° C. to about 0° C. In a further aspect, the styrene butadiene rubber can include a blend of two or more styrene-butadiene rubber having different T_{g}. In a further aspect, such a blend of styrene-butadiene rubbers can include functionalized SBRs, non-functionalized SBRs, or a combination of functionalized and non-functionalized styrene-butadiene rubbers.

In an aspect, commonly employed silicas that can be used in the disclosed rubber compositions include conventional pyrogenic and precipitated silica. In a further aspect, the precipitated silicas can be, for example, those obtained by the acidification of a soluble silicate, e.g., sodium silicate. In a further aspect, such conventional silicas can be characterized, for example, by having a BET surface area, as measured using nitrogen gas. In one embodiment, the BET surface area can be in the range of from about 150 square meters per gram to about 200 square meters per gram. In another embodiment, the BET surface area can be in a range of from about 160 square meters per gram to about 170 square meters per gram. In another aspect, the conventional silica can also be characterized by having a dibutylphthalate (DBP) absorption value in a range of from about 100 to about 400, alternatively from about 150 to about 300. In still another aspect, the conventional silica might be expected to have an average ultimate particle size, for example, in the range of from about 0.01 micron to 0.05 micron as determined by electron microscope, although other sizes are also contemplated and should be considered disclosed.

In one aspect, an untreated silica is useful herein. In a further aspect, the untreated silica can be a prewashed, wet filter cake of precipitated silica having hydroxyl groups thereon. In a further aspect, the precipitated silica can be derived from rice husk ash or bagasse (sugar cane) ash or the like and need not be dried following washing and filtration of an acid-treated sodium silicate.

In some aspects, the untreated silica is diluted in water (or other aqueous medium that is predominantly water) to form an aqueous slurry. In one embodiment, the slurry can contain from 5-30 wt % silica, or at least 10 wt % silica, or another suitable dilution that is able to be pumped through a spray dryer. In any of these aspects, the pH of the slurry can be adjusted, e.g., to a pH of from about 6 to about 7.

In one aspect, a silica gel can be derived, for example, by hydrophobating a silica hydrogel with, for example, an organomercaptosilane and alkyl silane and drying the product.

In some aspects, the temperature of the slurry is raised to (or maintained at) a temperature at which the coupling agent reacts with the hydrated surface of the silica, but which is below the boiling point of water, e.g., at least 20° C., or at least 40° C., or at least 60° C., or up to 90° C., or up to 95° C., such as between about 80 and about 85° C. In a further aspect, the silica coupling agent is allowed to react with the silica for a time which is sufficient to allow completion of the reaction.

In some aspects, the slurry is then dried, e.g., by spray drying, at a temperature above the boiling point of water, such as at least 130° C. or at least 150° C. In one aspect, the dried product can be in the form of substantially spherical beads, granules, or a powder (all of which are generally referred to herein as particles).

In some aspects, an organosilane coupling agent is incorporated into the disclosed compositions. In one aspect, the organosilane coupling agent used can include a first reactive moiety which includes a silicon atom and at least one hydrolyzable group, and a second reactive moiety capable of reaction with a double bond of a vulcanizable elastomer, the first and second reactive moieties being connected by a bridging unit comprising at least one of a polysulfide and a hydrocarbylene group. Further in this aspect, at least one of the hydrolyzable groups of the first reactive moiety can be selected from an alkoxy group and an aminoalkyl group. In some aspects, the coupling agent can be selected from bis(trialkoxysilylalkyl) polysulfides, bis(alkoxyaryloxysilylalkyl) polysulfides, bis(triaryloxysilylalkyl) polysulfides, and mixtures thereof.

In one aspect, the pretreated silica material can have a CTAB surface area of at least 30 m²/g, or at least 50 m²/g, or at least 100 m²/g, or at least 200 m²/g, or up to 300 m²/g, or up to 400 m²/g, or up to 500 m²/g, as measured according to ASTM D6845-20. In one aspect, the untreated precipitated silica can have a pH of 6-8, resulting from residual sodium sulfate.

In one aspect, the untreated silica can have a particle size of less than 10 micrometers (μm), or less than 5 μm, or at least 0.1 μm, as determined by ASTM C721-20, “Standard Test Methods for Estimating Average Particle Size of Alumina and Silica Powders by Air Permeability.” (Silica primary particles for rubber usage are usually in the 10 to 100 nanometer range, 0.01 to 0.1 micrometers) as measured by Differential Centrifugal Sedimentation (DCS).

Exemplary untreated precipitated silicas are available from PPG Industries as Hi-Sil™, e.g., under the designations 210, 243, 315, EZ 160G-D, EZ 150G, 190G, 200G-D, HDP-320G, and 255CG-D; from Solvay as Zeosil™, under the designations 115GR, 125GR, 165GR, 175GR, 185GR, 195GR, 1085GR, 1165MP, 1115MP, HRS 1200MP, Premium MP, Premium 200MP, and 195 HR; from Evonik as Ultrasil™, under the designations VN2, VN3, VN3GR, 5000GR, 7000GR, 9000GR, as Zeopol™, under the designations 8755LS and 8745; from Wuxi Quechen Silicon Chemical Co., Ltd. as Newsil™, under the designations 115GR and 2000MP; from Maruo Calcium Co., Ltd., as Tokusil™ 315, and silicas derived from rice husk ash from Yihai Food and Oil Industry, China. Any precipitated silica can be used in the method. In other embodiments, the untreated precipitated silica is prepared as a wet filtered material shortly before use.

In one aspect, a silica coated carbon black and/or commonly employed carbon blacks can also be used as filler in an amount ranging from 10 to 150 phr. In another embodiment, from 20 to 80 phr of carbon black can be used. Representative examples of such carbon blacks include N110, N121, N134, N220, N231, N234, N242, N293, N299, N315, N326, N330, N332, N339, N343, N347, N351, N358, N375, N539, N550, N582, N630, N642, N650, N683, N754, N762, N765, N774, N787, N907, N908, N990 and N991. These carbon blacks have iodine absorptions ranging from 9 to 145 g/kg and DBP number ranging from 34 to 150 cm³/100 g.

In another aspect, other fillers can be used in the rubber composition including, but not limited to, particulate fillers including ultra-high molecular weight polyethylene (UHMWPE), crosslinked particulate polymer gels and plasticized starch composite filler. In a further aspect, such other fillers can be used in an amount ranging from 1 phr to 30 phr.

In another aspect, the rubber composition can optionally include rubber processing oil. Further in this aspect, the rubber composition can include from 0 to about 100 phr of processing oil. In a still further aspect, processing oil can be included in the rubber composition as extending oil typically used to extend elastomers. In another aspect, processing oil can also be included in the rubber composition by addition of the oil directly during rubber compounding. In yet another aspect, the processing oil used can include both extending oil present in the uncured rubber prior to processing, and process oil added during compounding. In one embodiment, the rubber composition includes a low PCA oil. Suitable low PCA oils include, but are not limited to, mild extraction solvates (MES), treated distillate aromatic extracts (TDAE), residual aromatic extract (RAE), SRAE, and heavy naphthenic oils as are known in the art. In a further aspect, suitable low PCA oils include those having a polycyclic aromatic content of less than 3 percent by weight as determined by the IP 346 method. Procedures for the IP 346 method can be found in Standard Methods for Analysis and Testing of Petroleum and Related Products and British Standard 2000 Parts, 2003, 62nd edition, published by the Institute of Petroleum, United Kingdom.

In an aspect, suitable TDAE oils are available as Tudalen® SX500 from Klaus Dahleke K G, VivaTec® 400 and VivaTec® 500 from H&R Group, Enerthene® 1849 from BP, and Extensoil® 1996 from Repsol. In a further aspect, the oils may be available as the oil alone or can be provided along with an elastomer in the form of an extended elastomer. In another aspect, suitable vegetable oils include, but are not limited to, soybean oil, sunflower oil, and canola oil, which can be provided in the form of esters containing a certain degree of unsaturation.

In one aspect, a rubber composition including the disclosed antioxidants and/or antiozonants could be compounded by methods generally known in the rubber compounding art, such as mixing various sulfur-vulcanizable constituent rubbers with variously commonly used additive materials such as, for example, curing aids, such as sulfur activators, retarders and accelerators, processing additives such as oils, resins including tackifying, traction, and thermoplastic resins and plasticizers, fillers, pigments, fatty acid, zinc acid, waxes, antioxidants and antiozonants (anti-degradants), peptizing agents, and reinforcing materials. In an aspect, depending on the intended use of the sulfur vulcanizable and sulfur vulcanized material (rubbers), the additives mentioned above are selected and commonly used in conventional amounts.

Representative examples of sulfur donors include elemental sulfur (free sulfur), an amine disulfide, polymeric polysulfide, and sulfur olefin adducts. In one aspect, the sulfur-vulcanizing agent is elemental sulfur. In another aspect, the sulfur-vulcanizing agent can be used in an amount ranging from about 0.5 phr to about 8 phr, or from about 1.5 phr to about 6 phr. Typical amounts of resins can be added in a range of from about 0 phr to about 100 phr. Typical amounts of processing aids can be added in an amount of from about 1 phr to about 50 phr. Typical amounts of antioxidants can be from about 1 phr to about 5 phr. In an aspect, representative antioxidants include, but are not limited to, diphenyl-p-phenylenediamine. Typical amounts of antiozonants can be from about 1 phr to about 5 phr. Typical amounts of fatty acids can include stearic acid and can be present in an amount of from about 0.5 phr to about 3 phr.

In one aspect, typical amounts of zinc oxide can be from about 2 phr to about 5 phr. Typical amounts of waxes can be from about 1 phr to about 5 phr. In an aspect, microcrystalline waxes can be used. Typical amounts of peptizers can be from about 0.1 phr to about 1 phr. In one aspect, a typical peptizer can be, for example, dibenzamidodiphenyl disulfide.

In one aspect, accelerators can be used to control the time and/or temperature required for vulcanization and to improve the properties of the vulcanizate. In one embodiment, a single accelerator system can be used, i.e., a primary accelerator. In another embodiment, the primary accelerator(s) can be used in total amounts ranging from about 0.5 phr to about 4 phr, or from about 0.8 phr to about 1.5, phr. In another embodiment, combinations of a primary and a secondary accelerator can be used, with the secondary accelerator being used in smaller amounts, such as from about 0.05 phr to about 3 phr, in order to activate and to improve the properties of the vulcanizate. In an aspect, combinations of these accelerators might be expected to produce a synergistic effect on the final properties of the cured rubber and may be somewhat better than those produced by use of either accelerator alone. In an additional aspect, delayed action accelerators can be used; these are not affected by normal processing temperatures but produce a satisfactory cure at ordinary vulcanization temperatures. In some aspects, vulcanization retarders can also be used. Suitable types of accelerators that can be used in the present invention are amines, disulfides, guanidines, thioureas, thiazoles, thiurams, sulfonamides, dithiocarbamates, and xanthates. In one embodiment, the primary accelerator is a sulfonamide. If a second accelerator is used, the secondary accelerator can be a guanidine, dithiocarbamate, or thiuram compound. In another aspect, other curatives can be used, including, but not limited to, from about 0.5 phr to about 5 phr of 1,6-bis(N,N′ dibenzylthio-carbamoyldithio)-hexane, which is available as Vulcuren from Lanxess.

In one aspect, the cured rubber compound that includes the antioxidants and/or antiozonants disclosed herein can be incorporated in a variety of rubber articles, including, for example, tire components, rubber belts, and hoses, among others. In one aspect, the tire component can be a tire tread such including at least one of tread cap and/or tread base rubber layer tire sidewall, tire carcass component, such as, for example, a carcass cord ply coat, tire sidewall stiffening insert, an apex adjacent to or spaced apart from a tire bead, wire coat, inner liner tire chafer and/or tire bead component. In a further aspect, the tread and/or tires can be built, shaped, molded and cured by various methods which will be readily apparent to those skilled in the art.

In one aspect, the disclosed antioxidants and/or antiozonants migrate or bloom from an inner surface to an outer surface of the article over time. In one aspect, the rate of migration of the compound from the inner surface to the outer surface of the article is within 10% of a rate of migration for a compound selected from listed compounds

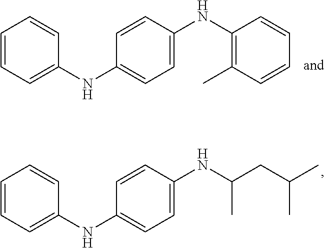 [SMILES: Cc1ccccc1Nc1ccc(Nc2ccccc2)cc1 ; CC(C)CC(C)Nc1ccc(Nc2ccccc2)cc1]

or is within a range from 10% above to 10% below the rate of migration of a listed compound, from 7.5% above to 7.5% below the rate of migration of a listed compound, from 5% above to 5% below the rate of migration of a listed compound, from 2.5% above to 2.5% below the rate of migration of listed compound, or has the same rate of migration as a listed compound.

In one aspect, a pneumatic tire as disclosed herein can be a race tire, passenger tire, aircraft tire, agricultural, earth-mover, off-the-road, truck tire, or the like. In one embodiment, the tire is a passenger or truck tire. In another embodiment, the tire can also be a radial or bias. In one embodiment, the tire component is intended to be ground-contacting. In another embodiment, the tire component is not ground contacting. In other embodiments, the rubber compound can be incorporated in a non-pneumatic tire.

In yet another aspect, the mixing of the rubber composition can be accomplished by methods known to those having skill in the rubber mixing art. For example, the ingredients are typically mixed in at least two stages, namely, at least one non-productive stage followed by a productive mix stage. Further in this aspect, the final curatives including sulfur-vulcanizing agents are typically mixed in the final stage which is conventionally called the “productive” mix stage in which the mixing typically occurs at a temperature, or ultimate temperature, lower than the mix temperature(s) than the preceding non-productive mix stage(s). The terms “non-productive” and “productive” mix stages are well known to those having skill in the rubber mixing art. In another aspect, the rubber composition can be subjected to a thermomechanical mixing step. Further in this aspect, the thermomechanical mixing step generally includes a mechanical working in a mixer or extruder for a period of time suitable in order to produce a rubber temperature between 140° C. and 190° C. The appropriate duration of the thermomechanical working varies as a function of the operating conditions, and the volume and nature of the components. In one example, the thermomechanical working can be from about 1 minute to about 20 minutes.

In still another aspect, after mixing, the compounded rubber can be fabricated by a method such as, for example, by extrusion through a suitable die to form a tire tread (including tread cap and tread base). In a further aspect, the tire tread is typically built onto a sulfur curable tire carcass and the assembly thereof cured in a suitable mold under conditions of elevated temperature and pressure by methods well-known to those having skill in the art.

Also disclosed herein is a method for enhancing at least one antiozonant property, at least one antioxidant property, or any combination thereof of a disclosed rubber composition, the method comprising incorporating a disclosed compound in the rubber composition.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. A compound of Formula I or a salt thereof:

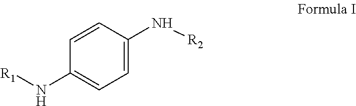

wherein R₁ and R₂ independently comprise linear or branched C3-C20 alkyl; substituted or unsubstituted C3-C20 cycloalkyl, linear or branched C3-C20 alkenyl; fused or bridged C3-C20 cycloalkyl, substituted or unsubstituted C₅-C₁₀ heteroaryl, or substituted or unsubstituted C₅-C₁₀ aryl;
provided that R₂ is not

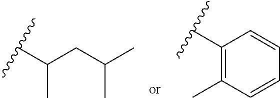

when R₁ is phenyl.

Aspect 2. The compound or salt of aspect 1, wherein R₁ comprises unsubstituted C₅-C₆ aryl or C₅-C₆ aryl substituted with a C₁-C₄ linear or branched alkyl group or a C₅-C₁₀ aryl group.

Aspect 3. The compound or salt of aspect 1 or 2, wherein R₁ and R₂ are different.

Aspect 4. The compound or salt of any one of aspects 1-3, wherein R₁ and R₂ are both aryl.

Aspect 5. The compound or salt of any one of aspects 1-3, wherein R₁ is aryl and R₂ is alkyl or cycloalkyl.

Aspect 6. The compound or salt of any one of aspects 1-3, wherein R₁ and R₂ are both alkyl, both cycloalkyl, or wherein one of R₁ and R₂ is alkyl and one of R₁ and R₂ is cycloalkyl.

Aspect 7. The compound or salt of aspect 1, having the structure:

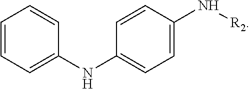

Aspect 8. The compound or salt of any one of aspects 1-7, wherein R₂ is a C4-C20 alkyl group.

Aspect 9. The compound or salt of any one of aspects 1-8, wherein R₂ is selected from

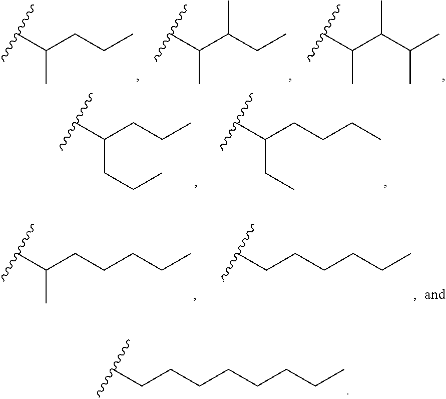

Aspect 10. The compound or salt of any one of aspects 1-7, wherein R₂ is a substituted or unsubstituted C3-C20 cycloalkyl group.

Aspect 11. The compound or salt of any one of aspects 1-7, wherein R₂ is selected from

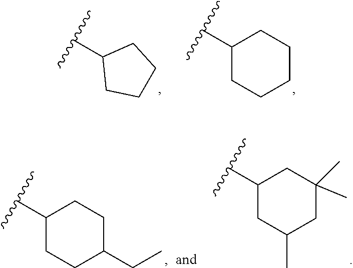

Aspect 12. The compound or salt of any one of aspects 1-7, wherein R₂ is a linear or branched C3-C10 alkenyl group comprising from 1-3 lower alkyl substituents on one double bond.

Aspect 13. The compound of any one of aspects 1-7, wherein R₂ is

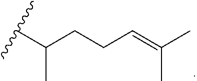 [SMILES: *C(C)CCC=C(C)C]

Aspect 14. The compound or salt of any one of aspects 1-7, wherein R₂ is a fused or bridged C3-C20 cycloalkyl group.

Aspect 15. The compound or salt of any one of aspects 1-7, wherein R₂ is a fused or bridged cycloalkyl group selected from adamantyl, norbornyl, or decalyl.

Aspect 16. The compound or salt of any one of aspects 1-7, wherein R₂ is

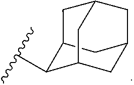 [SMILES: *C1C2CC3CC(C2)C(C)C1C3]

Aspect 17. The compound or salt of any one of aspects 1-16, wherein the compound is selected from

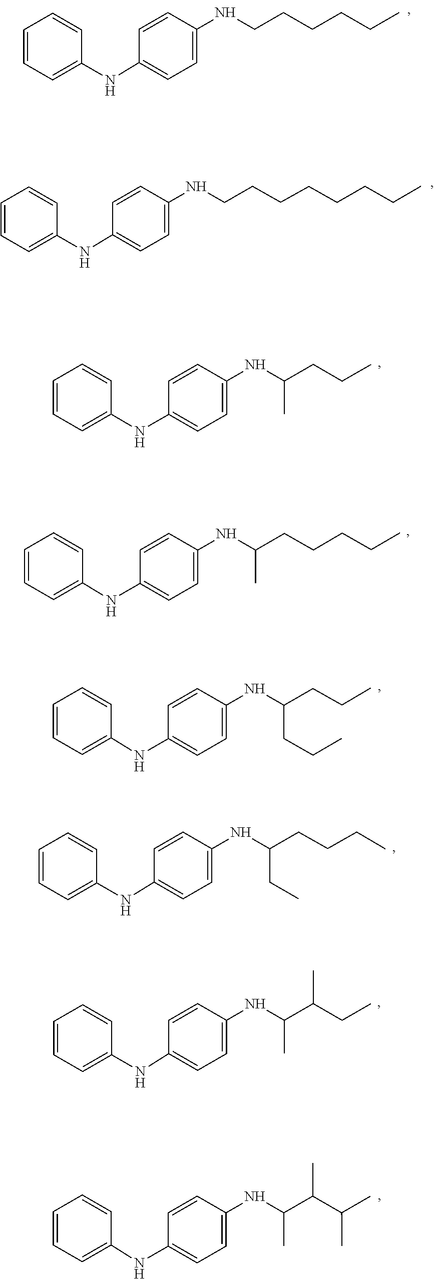

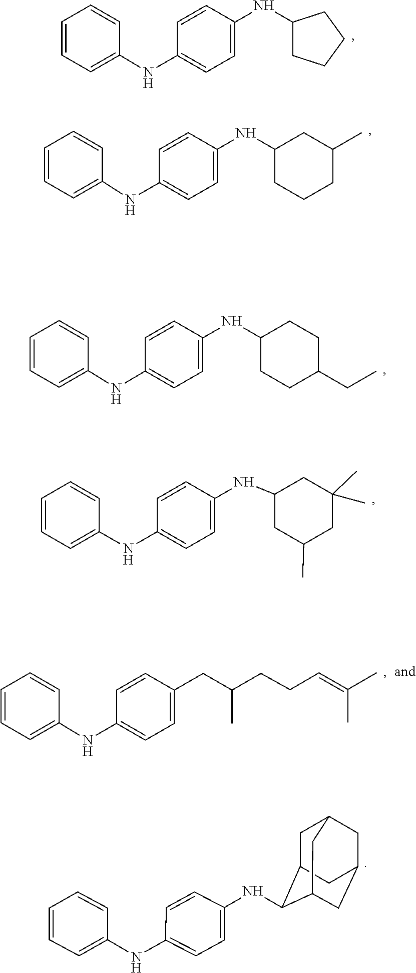 [SMILES: c1ccc(Nc2ccc(NC3CCCC3)cc2)cc1 ; CC1CCCC(Nc2ccc(Nc3ccccc3)cc2)C1 ; CCC1CCC(Nc2ccc(Nc3ccccc3)cc2)CC1 ; CC1CC(Nc2ccc(Nc3ccccc3)cc2)CC(C)(C)C1 ; CC(C)=CCCC(C)Cc1ccc(Nc2ccccc2)cc1 ; c1ccc(Nc2ccc(NC3C4CC5CC(C4)CC3C5)cc2)cc1]

Aspect 18. The salt of any one of aspects 1-17, wherein the salt has a counter-ion selected from Na⁺, K⁺, Ca²⁺, Mg²⁺, Zn²⁺, or any combination thereof.

Aspect 19. The compound or salt of any one of aspects 1-18, wherein the compound and its degradation products are non-toxic.

Aspect 20. A method for making the compound or salt of any one of aspects 1-19, the method comprising admixing a compound of Formula II

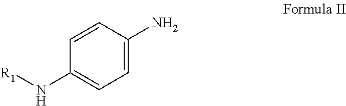

with a carbonyl compound comprising an aldehyde or ketone having Formula III and a reducing agent

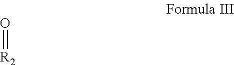

wherein R₁ and R₂ independently comprise linear or branched C3-C20 alkyl; substituted or unsubstituted C3-C20 cycloalkyl, linear or branched C3-C10 alkenyl; fused or bridged C3-C20 cycloalkyl, or substituted or unsubstituted C₅-C₁₀ aryl;
provided that R₂ is not

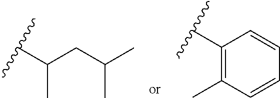

when R₁ is phenyl.

Aspect 21. The method of aspect 20, wherein the compound of Formula I is admixed with the carbonyl compound to produce a first composition, then the first composition is admixed with a reducing agent, optionally in the presence of acetic acid.

Aspect 22. The method of aspect 20 or 21, wherein the method further comprises treating the compound with a base to form the salt.

Aspect 23. The method of aspect 22, wherein the base comprises NaOH, KOH, Ca(OH)₂, Mg(OH)₂, Zn(OH)₂, CaO, K₂CO₃, Na₂CO₃, Na(0), or any combination thereof.

Aspect 24. The method of any one of aspects 20-23, wherein the reducing agent comprises NaBH(OAc)₃, NaBH₄, Na(CN)BH₃, or any combination thereof.

Aspect 25. The method of any one of aspects 20-24, wherein the method is conducted in a polar aprotic solvent.

Aspect 26. The method of aspect 25, wherein the solvent comprises tetrahydrofuran (THF), toluene, xylene, or any combination thereof.

Aspect 27. A method for making the compound or salt of any one of aspects 1-19, the method comprising admixing hydroquinone with R₁—NH₂, R₂—NH₂, and a Lewis acid in a solvent.

Aspect 28. The method of aspect 27, wherein the Lewis acid comprises FeCl₃.

Aspect 29. The method of aspect 27 or 28, wherein the solvent comprises tetrahydrofuran (THF), toluene, xylene, or any combination thereof.

Aspect 30. A method for making the compound or salt of any one of aspects 1-19, the method comprising:
(a) admixing a compound having a structure of Formula II

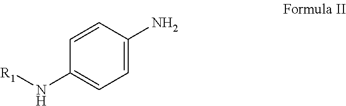

with an aldehyde or ketone having Formula III

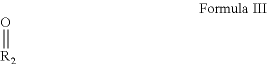

in a solvent to form an intermediate; and
(b) performing catalytic hydrogenation on the intermediate to produce the compound or salt;
wherein R₁ and R₂ independently comprise linear or branched C3-C20 alkyl; substituted or unsubstituted C3-C20 cycloalkyl, linear or branched C3-C20 alkenyl; fused or bridged C3-C20 cycloalkyl, substituted or unsubstituted C₅-C₁₀ heteroaryl, or substituted or unsubstituted C₅-C₁₀ aryl;
provided that R₂ is not

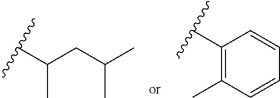

when R₁ is phenyl.

Aspect 31. The method of aspect 30, wherein the solvent comprises tetrahydrofuran (THF), toluene, xylene, or any combination thereof.

Aspect 32. The method of aspect 30 or 31, wherein catalytic hydrogenation is performed using platinum dioxide, cobalt boride, Crabtree's catalyst, HRh[P(C₆H₅)₃]₄, Lindlar catalyst, nickel boride, palladium black, palladium on carbon, Raney nickel, Rh—Pt oxide, Urushibara nickel, Wilkinson's catalyst, or any combination thereof.

Aspect 33. A rubber composition comprising one or more rubber polymers and the compound or salt of any one of aspects 1-19.

Aspect 34. The rubber composition of aspect 33, wherein the one or more rubber polymers comprise natural rubber, polybutadiene, styrene-butadiene rubber, nitrile rubber, polyisoprene, chloroprene, ethylene-butadiene, hydrogenated butadiene, hydrogenated styrene-butadiene, or any combination thereof.

Aspect 35. The rubber composition of any one of aspects 33 or 34, wherein the compound has at least one antiozonant property, at least one antioxidant property, or any combination thereof.

Aspect 36. The rubber composition of aspect 35, wherein the antiozonant property, the antioxidant property, or both comprise prevention of cracking of the one or more rubber polymers, slowing of cracking of the one or more rubber polymers, prevention of a color change of the one or more rubber polymers, preservation of flexibility of the one or more rubber polymers, or any combination thereof.

Aspect 37. An article comprising the rubber composition of any one of aspects 33-36.

Aspect 38. The article of aspect 37, wherein the article is a tire.

Aspect 39. The article of aspect 37 or 38, wherein the compound migrates from an inner surface to an outer surface of the article over time.

Aspect 40. The article of aspect 39, wherein a rate of migration of the compound from the inner surface to the outer surface of the article is within 10% of a rate of migration for a compound selected from

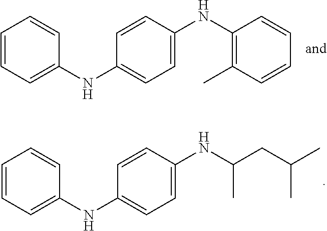 [SMILES: Cc1ccccc1Nc1ccc(Nc2ccccc2)cc1 ; CC(C)CC(C)Nc1ccc(Nc2ccccc2)cc1]

Aspect 41. A method for enhancing at least one antiozonant property, at least one antioxidant property, or any combination thereof of a rubber composition, the method comprising incorporating the compound or salt of any one of aspects 1-19 in a rubber composition.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Synthetic Schemes

Non-limiting synthetic schemes using a paraphenylenediamine starting material with an a ketone or aldehyde reaction are shown in Schemes 1 and 2 below. Similar reactions can be performed to make different products by varying substituents on the paraphenylenediamine analog starting material.

In some experiments, for compounds using a ketone as a side-chain donor, the following general reaction scheme was used:

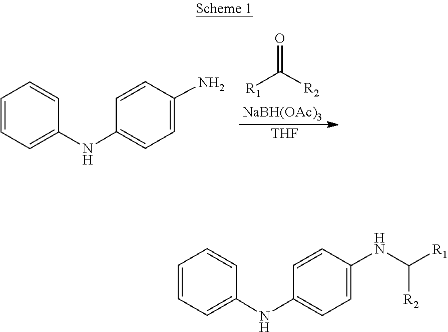

To a 2 L 3-neck flask, paraphenylenediamine (20 g) was dissolved in THF (400 mL) and added the respective ketone (9.6-18 mL). Sodium triacetoxyborohydride (25.3 g) was added followed by acetic acid (6.2 mL). The inner atmosphere was flushed with nitrogen. The reaction progress was monitored via TLC and GCMS. After 24-96 h, the reaction was quenched with aqueous 1 M NaOH solution (400 mL). The organic layer was the subsequently washed with additional 1 M NaOH and brine. The organic layer was dried over magnesium sulfate and concentrated. The crude product was either purified via recrystallization in hexane or flash column chromatography using ethyl acetate/hexane eluent.

In some experiments, for compounds using an aldehyde as a side-chain donor, the following general reaction scheme was used:

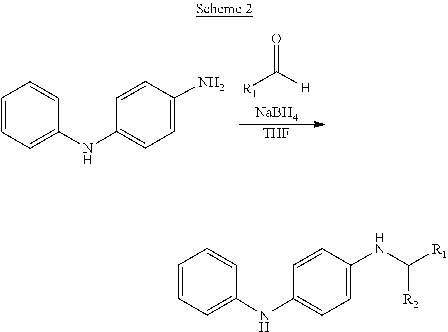

Thus, synthesis was performed without acetic acid when an aldehyde was used in place of a ketone (e.g., hexanal or octanal).

### Example 2: Ozone Test

Rubber compositions were mixed to include 4 phr of disclosed antioxidant/antiozonant compounds and cured at 170° C. for 10 min. Rubber compositions were cut into strips and placed into an ozone chamber. The strips were attached to a die and constantly vibrated. Ozone (50 ppm) was cycled in the chamber for 4 hours and then shut off for 4 hours. This 8 hour cycle was repeated until 21 days had elapsed and the samples were visually inspected for cracks, discoloration, and breakage. 6PPD was used as a positive control and N,N′-diphenyl-p-phenylenediamine (DPPD) was used as a negative control. Compounds tested are shown in Table 1. Testing results are presented in Tables 2-5. Repeat columns in a table indicate replicates of the same disclosed antioxidant/antiozonant as indicated by the column heading. Results key: BRK=sample was BRK completely. Fraction+SRF=density of cracks covered approximately the given fraction of the surface. Severity of cracks=depth of cracks into surface given by measurements in tables.

TABLE 1
Tested Compounds

| Compound Identifier | Structure |
|---|---|
| IPPD | 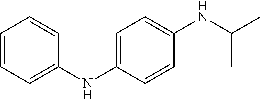 [SMILES: CC(C)Nc1ccc(Nc2ccccc2)cc1] |
| A | 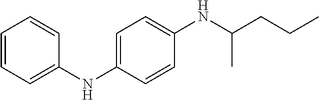 [SMILES: CCCC(C)Nc1ccc(Nc2ccccc2)cc1] |
| B | 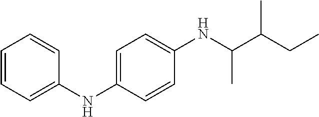 [SMILES: CCC(C)C(C)Nc1ccc(Nc2ccccc2)cc1] |
| C | 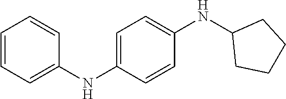 [SMILES: c1ccc(Nc2ccc(NC3CCCC3)cc2)cc1] |
| D | 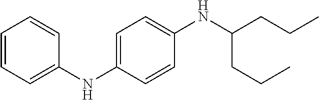 [SMILES: CCCC(CCC)Nc1ccc(Nc2ccccc2)cc1] |
| E | 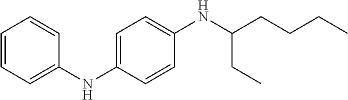 [SMILES: CCCCC(CC)Nc1ccc(Nc2ccccc2)cc1] |
| F | 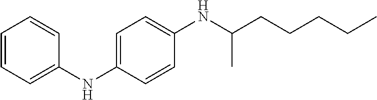 [SMILES: CCCCCC(C)Nc1ccc(Nc2ccccc2)cc1] |
| H | 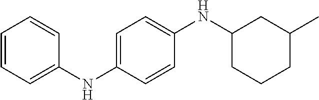 [SMILES: CC1CCCC(Nc2ccc(Nc3ccccc3)cc2)C1] |
| I | 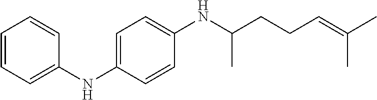 [SMILES: CC(C)=CCCC(C)Nc1ccc(Nc2ccccc2)cc1] |
| J | 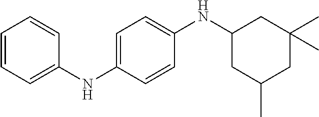 [SMILES: CC1CC(Nc2ccc(Nc3ccccc3)cc2)CC(C)(C)C1] |
| L | 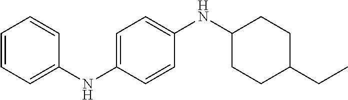 [SMILES: CCC1CCC(Nc2ccc(Nc3ccccc3)cc2)CC1] |
| N | 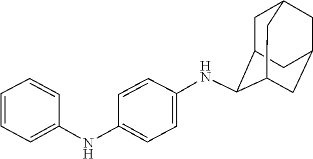 [SMILES: c1ccc(Nc2ccc(NC3C4CC5CC(C4)CC3C5)cc2)cc1] |
| Q | 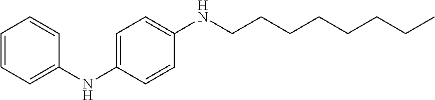 [SMILES: CCCCCCCCNc1ccc(Nc2ccccc2)cc1] |
| CHPD | 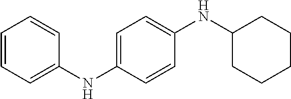 [SMILES: c1ccc(Nc2ccc(NC3CCCCC3)cc2)cc1] |

TABLE 2
Ozone Testing of Selected Compounds

| | DPPD | 6PPD | A | A | F | F | E | E | IPPD |
|---|---|---|---|---|---|---|---|---|---|
| Day 01 | NONE | NONE | NONE | NONE | NONE | NONE | NONE | NONE | NONE |
| DENSITY | | | | | | | | | |
| Day 01 | NONE | NONE | NONE | NONE | NONE | NONE | NONE | NONE | NONE |
| SEVERITY | | | | | | | | | |
| Day 02 | NONE | NONE | NONE | NONE | NONE | NONE | NONE | NONE | NONE |
| DENSITY | | | | | | | | | |
| Day 02 | NONE | NONE | NONE | NONE | NONE | NONE | NONE | NONE | NONE |
| SEVERITY | | | | | | | | | |
| Day 03 | NONE | NONE | NONE | NONE | NONE | NONE | NONE | NONE | NONE |
| DENSITY | | | | | | | | | |
| Day 03 | NONE | NONE | NONE | NONE | NONE | NONE | NONE | NONE | NONE |
| SEVERITY | | | | | | | | | |
| Day 04 | NONE | NONE | NONE | NONE | NONE | NONE | NONE | NONE | NONE |
| SEVERITY | | | | | | | | | |
| Day 04 | NONE | NONE | NONE | NONE | NONE | NONE | NONE | NONE | NONE |
| DENSITY | | | | | | | | | |
| Day 07 | 1/8 | NONE | NONE | NONE | NONE | NONE | NONE | NONE | NONE |
| DENSITY | SRF | | | | | | | | |
| Day 07 | 0.01 | NONE | NONE | NONE | NONE | NONE | NONE | NONE | NONE |
| SEVERITY | INCH | | | | | | | | |
| Day 08 | 1/8 | NONE | NONE | NONE | NONE | NONE | NONE | NONE | NONE |
| DENSITY | SRF | | | | | | | | |
| Day 08 | 0.01 | NONE | NONE | NONE | NONE | NONE | NONE | NONE | NONE |
| SEVERITY | INCH | | | | | | | | |
| Day 09 | 1/8 | NONE | NONE | NONE | NONE | NONE | NONE | NONE | NONE |
| DENSITY | SRF | | | | | | | | |
| Day 09 | 0.01 | NONE | NONE | NONE | NONE | NONE | NONE | NONE | NONE |
| SEVERITY | INCH | | | | | | | | |
| Day 10 | 0.01 | NONE | NONE | NONE | NONE | NONE | NONE | NONE | NONE |
| SEVERITY | INCH | | | | | | | | |
| Day 10 | 1/8 | NONE | NONE | NONE | NONE | NONE | NONE | NONE | NONE |
| DENSITY | SRF | | | | | | | | |
| Day 11 | 0.01 | NONE | NONE | NONE | NONE | NONE | NONE | NONE | NONE |
| SEVERITY | INCH | | | | | | | | |
| Day 11 | 3/0 | NONE | NONE | NONE | NONE | NONE | NONE | NONE | NONE |
| DENSITY | SRF | | | | | | | | |
| Day 14 | 0.01 | NONE | NONE | NONE | NONE | NONE | NONE | NONE | NONE |
| SEVERITY | INCH | | | | | | | | |
| Day 14 | 1/8 | NONE | NONE | NONE | NONE | NONE | NONE | NONE | NONE |
| DENSITY | SRF | | | | | | | | |
| Day 15 | 0.01 | NONE | NONE | NONE | NONE | NONE | NONE | NONE | NONE |
| SEVERITY | INCH | | | | | | | | |
| Day 15 | 1/8 | NONE | NONE | NONE | NONE | NONE | NONE | NONE | NONE |
| DENSITY | SRF | | | | | | | | |
| Day 16 | 1/8 | NONE | NONE | NONE | NONE | NONE | NONE | NONE | NONE |
| DENSITY | SRF | | | | | | | | |
| Day 16 | 0.01 | NONE | NONE | NONE | NONE | NONE | NONE | NONE | NONE |
| SEVERITY | INCH | | | | | | | | |
| Day 17 | 1/8 | NONE | NONE | NONE | NONE | NONE | NONE | NONE | NONE |
| DENSITY | SRF | | | | | | | | |
| Day 17 | 0.01 | NONE | NONE | NONE | NONE | NONE | NONE | NONE | NONE |
| SEVERITY | INCH | | | | | | | | |
| Day 18 | 1/8 | NONE | NONE | NONE | NONE | NONE | NONE | NONE | NONE |
| DENSITY | SRF | | | | | | | | |
| Day 18 | 0.01 | NONE | NONE | NONE | NONE | NONE | NONE | NONE | NONE |
| SEVERITY | INCH | | | | | | | | |
| Day 21 | 0.03 | 0.01 | 0.01 | 0.3 | 0.01 | 0.01 | 0.01 | 0.01 | 0.01 |
| SEVERITY | INCH | INCH | INCH | INCH | INCH | INCH | INCH | INCH | INCH |
| Day 21 | 3/4 | EDGE | EDGE | 3/4 | EDGE | EDGE | EDGE | 3/4 | 3/4 |
| DENSITY | SRF | | | SRF | | | | SRF | SRF |

TABLE 3
Ozone Testing of Selected Compounds

| | DPPD | 6PPD | B | B | C | C | D | D | IPPD | CHPD |
|---|---|---|---|---|---|---|---|---|---|---|
| Day 01 | 0.01 | NONE | NONE | NONE | NONE | NONE | NONE | NONE | NONE | NONE |
| SEVERITY | INCH | | | | | | | | | |
| Day 01 | EDGE | NONE | NONE | NONE | NONE | NONE | NONE | NONE | NONE | NONE |
| DENSITY | | | | | | | | | | |
| Day 02 | 0.01 | NONE | NONE | NONE | NONE | NONE | NONE | NONE | NONE | NONE |
| SEVERITY | INCH | | | | | | | | | |
| Day 02 | 1/8 | NONE | NONE | NONE | NONE | NONE | NONE | NONE | NONE | NONE |
| DENSITY | SRF | | | | | | | | | |
| Day 03 | 0.01 | NONE | NONE | NONE | NONE | NONE | NONE | NONE | NONE | NONE |
| SEVERITY | INCH | | | | | | | | | |
| Day 03 | 1/8 | NONE | NONE | NONE | NONE | NONE | NONE | NONE | NONE | NONE |
| DENSITY | SRF | | | | | | | | | |
| Day 04 | 0.01 | NONE | NONE | NONE | NONE | NONE | NONE | NONE | NONE | NONE |
| SEVERITY | INCH | | | | | | | | | |
| Day 04 | 1/8 | NONE | NONE | NONE | NONE | NONE | NONE | NONE | NONE | NONE |
| DENSITY | SRF | | | | | | | | | |
| Day 07 | 0.03 | 0.01 | 0.01 | 0.01 | 0.01 | 0.01 | 0.01 | 0.01 | 0.01 | 0.01 |
| SEVERITY | INCH | INCH | INCH | INCH | INCH | INCH | INCH | INCH | INCH | INCH |
| Day 07 | 1/8 | EDGE | EDGE | EDGE | EDGE | EDGE | EDGE | EDGE | EDGE | EDGE |
| DENSITY | SRF | | | | | | | | | |
| Day 08 | 0.03 | 0.01 | 0.01 | 0.01 | 0.01 | 0.01 | 0.01 | 0.01 | 0.01 | 0.01 |
| SEVERITY | INCH | INCH | INCH | INCH | INCH | INCH | INCH | INCH | INCH | INCH |
| Day 08 | 1/8 | EDGE | EDGE | EDGE | EDGE | EDGE | EDGE | 1/8 | EDGE | 1/8 |
| DENSITY | SRF | | | | | | | SRF | | SRF |
| Day 09 | 0.03 | 0.01 | 0.01 | 0.01 | 0.01 | 0.01 | 0.01 | 0.01 | 0.01 | 0.01 |
| SEVERITY | INCH | INCH | INCH | INCH | INCH | INCH | INCH | INCH | INCH | INCH |
| Day 09 | 1/8 | EDGE | EDGE | EDGE | EDGE | EDGE | EDGE | 1/8 | EDGE | 1/8 |
| DENSITY | SRF | | | | | | | SRF | | SRF |
| Day 10 | BRK | 0.10 | 0.01 | 0.10 | 0.01 | 0.01 | 0.01 | 0.01 | 0.01 | 0.01 |
| SEVERITY | | INCH | INCH | INCH | INCH | INCH | INCH | INCH | INCH | INCH |
| Day 10 | BRK | 1/8 | 1/8 | 1/8 | 1/8 | 1/8 | 1/8 | 1/8 | 1/8 | 1/8 |
| DENSITY | | SRF | SRF | SRF | SRF | SRF | SRF | SRF | SRF | SRF |
| Day 11 | BRK | 0.25 | BRK | BRK | 0.10 | 0.10 | 0.10 | 0.10 | 0.25 | 0.03 |
| SEVERITY | | INCH | | | INCH | INCH | INCH | INCH | INCH | INCH |
| Day 11 | BRK | 3/4 | BRK | BRK | 3/8 | 3/8 | 3/8 | 3/8 | 5/8 | 3/8 |
| DENSITY | | SRF | | | SRF | SRF | SRF | SRF | SRF | SRF |
| Day 14 | BRK | BRK | BRK | BRK | BRK | BRK | BRK | BRK | BRK | 0.10 |
| SEVERITY | | | | | | | | | | INCH |
| Day 14 | BRK | BRK | BRK | BRK | BRK | BRK | BRK | BRK | BRK | 3/8 |
| DENSITY | | | | | | | | | | SRF |
| Day 15 | BRK | BRK | BRK | BRK | BRK | BRK | BRK | BRK | BRK | 0.25 |
| SEVERITY | | | | | | | | | | INCH |
| Day 15 | BRK | BRK | BRK | BRK | BRK | BRK | BRK | BRK | BRK | 1/4 |
| DENSITY | | | | | | | | | | SRF |
| Day 16 | BRK | BRK | BRK | BRK | BRK | BRK | BRK | BRK | BRK | 0.25 |
| SEVERITY | | | | | | | | | | INCH |
| Day 16 | BRK | BRK | BRK | BRK | BRK | BRK | BRK | BRK | BRK | 3/4 |
| DENSITY | | | | | | | | | | SRF |
| Day 17 | BRK | BRK | BRK | BRK | BRK | BRK | BRK | BRK | BRK | 0.25 |
| SEVERITY | | | | | | | | | | INCH |
| Day 17 | BRK | BRK | BRK | BRK | BRK | BRK | BRK | BRK | BRK | 3/4 |
| DENSITY | | | | | | | | | | SRF |
| Day 18 | BRK | BRK | BRK | BRK | BRK | BRK | BRK | BRK | BRK | BRK |
| SEVERITY | | | | | | | | | | |
| Day 18 | BRK | BRK | BRK | BRK | BRK | BRK | BRK | BRK | BRK | BRK |
| DENSITY | | | | | | | | | | |
| Day 21 | BRK | BRK | BRK | BRK | BRK | BRK | BRK | BRK | BRK | BRK |
| SEVERITY | | | | | | | | | | |
| Day 21 | BRK | BRK | BRK | BRK | BRK | BRK | BRK | BRK | BRK | BRK |
| DENSITY | | | | | | | | | | |

TABLE 4
Ozone Testing of Selected Compounds

| | DPPD | 6PPD | I | I | J | J | N | N | CHPD |
|---|---|---|---|---|---|---|---|---|---|
| DAY 01 | NONE | NONE | NONE | NONE | NONE | NONE | NONE | NONE | NONE |
| SEVERITY | | | | | | | | | |
| DAY 01 | NONE | NONE | NONE | NONE | NONE | NONE | NONE | NONE | NONE |
| DENSITY | | | | | | | | | |
| DAY 02 | NONE | NONE | NONE | NONE | NONE | NONE | NONE | NONE | NONE |
| SEVERITY | | | | | | | | | |
| DAY 02 | NONE | NONE | NONE | NONE | NONE | NONE | NONE | NONE | NONE |
| DENSITY | | | | | | | | | |
| DAY 03 | NONE | NONE | NONE | NONE | NONE | NONE | NONE | NONE | NONE |
| SEVERITY | | | | | | | | | |
| DAY 03 | NONE | NONE | NONE | NONE | NONE | NONE | NONE | NONE | NONE |
| DENSITY | | | | | | | | | |
| DAY 06 | BRK | 0.01 | 0.01 | 0.01 | 0.01 | 0.01 | 0.01 | 0.01 | 0.01 |
| SEVERITY | | INCH | INCH | INCH | INCH | INCH | INCH | INCH | INCH |
| DAY 06 | BRK | ED GE | ED GE | ED GE | EDGE | EDGE | EDGE | EDGE | EDGE |
| DENSITY | | | | | | | | | |
| DAY 07 | BRK | 0.01 | 0.01 | 0.01 | 0.01 | 0.01 | 0.01 | 0.01 | 0.01 |
| SEVERITY | | INCH | INCH | INCH | INCH | INCH | INCH | INCH | INCH |
| DAY 07 | BRK | ED GE | 18 | EDGE | EDGE | EDGE | EDGE | EDGE | 1/8 |
| DENSITY | | | SRF | | | | | | SRF |
| DAY 08 | BRK | 0.01 | 0.01 | 0.01 | 0.01 | 0.01 | 0.01 | 0.01 | 0.01 |
| SEVERITY | | INCH | INCH | INCH | INCH | INCH | INCH | INCH | INCH |
| DAY 08 | BRK | ED GE | 1/8 | ED GE | EDGE | EDGE | EDGE | 1/8 | EDGE |
| DENSITY | | | SRF | | | | | SRF | |
| DAY 09 | BRK | BRK | BRK | BRK | 0.01 | 0.01 | 0.01 | 0.01 | 0.01 |
| SEVERITY | | | | | INCH | INCH | INCH | INCH | INCH |
| DAY 09 | BRK | BRK | BRK | BRK | 3/4 | 3/4 | 3/4 | 3/4 | 3/4 |
| DENSITY | | | | | SRF | SRF | SRF | SRF | SRF |
| DAY 10 | BRK | BRK | BRK | BRK | 0.01 | 0.01 | 0.01 | 0.01 | 0.01 |
| SEVERITY | | | | | INCH | INCH | INCH | NCH | INCH |
| DAY 10 | BRK | BRK | BRK | BRK | 3/4 | 3/4 | 3/4 | 3/4 | 3/4 |
| DENSITY | | | | | SRF | SRF | SRF | SRF | SRF |
| DAY 11 | BRK | BRK | BRK | BRK | 0.01 | 0.01 | 0.01 | 0.01 | 0.01 |
| SEVERITY | | | | | INCH | INCH | INCH | INCH | INCH |
| DAY 11 | BRK | BRK | BRK | BRK | 3/4 | 3/4 | 3/4 | 3/4 | 3/4 |
| DENSITY | | | | | SRF | SRF | SRF | SRF | SRF |
| DAY 12 | BRK | BRK | BRK | BRK | 0.01 | 0.01 | 0.01 | 0.01 | 0.01 |
| SEVERITY | | | | | INCH | INCH | INCH | INCH | INCH |
| DAY 12 | BRK | BRK | BRK | BRK | 3/4 | 3/4 | 3/4 | 3/4 | 3/4 |
| DENSITY | | | | | SRF | SRF | SRF | SRF | SRF |
| DAY 13 | BRK | BRK | BRK | BRK | 0.01 | 0.01 | 0.01 | 0.01 | 0.01 |
| SEVERITY | | | | | INCH | INCH | INCH | INCH | INCH |
| DAY 13 | BRK | BRK | BRK | BRK | 3/4 | 3/4 | 3/4 | 3/4 | 3/4 |
| DENSITY | | | | | SRF | SRF | SRF | SRF | SRF |
| DAY 16 | BRK | BRK | BRK | BRK | 0.25 | 0.01 | 0.01 | 0.01 | BRK |
| SEVERITY | | | | | INCH | INCH | INCH | INCH | |
| DAY 16 | BRK | BRK | BRK | BRK | 3/4 | 3/4 | 3/4 | 3/4 | BRK |
| DENSITY | | | | | SRF | SRF | SRF | SRF | |
| DAY 17 | BRK | BRK | BRK | BRK | 0.1 | 0.25 | 0.1 | 0.1 | BRK |
| SEVERITY | | | | | INCH | INCH | INCH | INCH | |
| DAY 17 | BRK | BRK | BRK | BRK | 3/4 | 3/4 | 3/4 | 3/4 | BRK |
| DENSITY | | | | | SRF | SRF | SRF | SRF | |
| DAY 18 | BRK | BRK | BRK | BRK | 0.25 | 0.1 | 0.1 | 0.1 | BRK |
| SEVERITY | | | | | INCH | INCH | INCH | INCH | |
| DAY 18 | BRK | BRK | BRK | BRK | 3/4 | 3/4 | 3/4 | 3/4 | BRK |
| DENSITY | | | | | SRF | SRF | SRF | SRF | |
| DAY 19 | BRK | BRK | BRK | BRK | BRK | 0.1 | 0.1 | 0.1 | BRK |
| SEVERITY | | | | | | INCH | INCH | INCH | |
| DAY 19 | BRK | BRK | BRK | BRK | BRK | 3/4 | 3/4 | 3/4 | BRK |
| DENSITY | | | | | | SRF | SRF | SRF | |
| DAY 20 | BRK | BRK | BRK | BRK | BRK | 0.1 | 0.1 | BRK | BRK |
| SEVERITY | | | | | | INCH | INCH | | |
| DAY 20 | BRK | BRK | BRK | BRK | BRK | 3/4 | 3/4 | BRK | BRK |
| DENSITY | | | | | | SRF | SRF | | |
| DAY 21 | BRK | BRK | BRK | BRK | BRK | 0.1 | 0.1 | BRK | BRK |
| SEVERITY | | | | | | INCH | INCH | | |
| DAY 21 | BRK | BRK | BRK | BRK | BRK | 3/4 | 3/4 | BRK | BRK |
| DENSITY | | | | | | SRF | SRF | | |

TABLE 5
Ozone Testing of Selected Compounds

| | DPPD | 6PPD | H | H | L | L | Q | Q | IPPD |
|---|---|---|---|---|---|---|---|---|---|
| DAY 01 | NONE | NONE | NONE | NONE | NONE | NONE | NONE | NONE | NONE |
| SEVERITY | | | | | | | | | |
| DAY 01 | NONE | NONE | NONE | NONE | NONE | NONE | NONE | NONE | NONE |
| DENSITY | | | | | | | | | |
| DAY 02 | NONE | NONE | NONE | NONE | NONE | NONE | NONE | NONE | NONE |
| SEVERITY | | | | | | | | | |
| DAY 02 | NONE | NONE | NONE | NONE | NONE | NONE | NONE | NONE | NONE |
| DENSITY | | | | | | | | | |
| DAY 03 | 0.01 | NONE | NONE | NONE | NONE | NONE | NONE | NONE | NONE |
| SEVERITY | INCH | | | | | | | | |
| DAY 03 | 1/8 | NONE | NONE | NONE | 1/8 | NONE | NONE | NONE | NONE |
| DENSITY | SRF | | | | SRF | | | | |
| DAY 04 | 0.01 | NONE | NONE | NONE | 0.01 | NONE | NONE | NONE | NONE |
| SEVERITY | INCH | | | | INCH | | | | |
| DAY 04 | 1/8 | NONE | NONE | NONE | 1/8 | NONE | NONE | NONE | NONE |
| DENSITY | SRF | | | | SRF | | | | |
| DAY 07 | 0.01 | 0.01 | 0.01 | 0.01 | 0.01 | 0.01 | 0.01 | 0.01 | NONE |
| SEVERITY | INCH | INCH | INCH | INCH | INCH | INCH | INCH | INCH | |
| DAY 07 | 3/8 | EDGE | EDGE | EDGE | 1/8 | EDGE | EDGE | EDGE | NONE |
| DENSITY | SRF | | | | SRF | | | | |
| DAY 08 | 0.01 | 0.01 | 0.01 | 0.01 | 0.01 | 0.01 | 0.01 | 0.01 | NONE |
| SEVERITY | INCH | INCH | INCH | INCH | INCH | INCH | INCH | INCH | |
| DAY 08 | 3/8 | EDGE | EDGE | EDGE | 1/8 | EDGE | EDGE | EDGE | NONE |
| DENSITY | SRF | | | | SRF | | | | |
| DAY 09 | 0.01 | 0.01 | 0.01 | 0.01 | 0.01 | 0.01 | 0.01 | 0.01 | 0.01 |
| SEVERITY | INCH | NCH | INCH | INCH | INCH | INCH | INCH | INCH | INCH |
| DAY 09 | 3/8 | EDGE | EDGE | 3/4 | 3/4 | 1/8 | 1/8 | 1/8 | EDGE |
| DENSITY | SRF | | | SRF | SRF | SRF | SRF | SRF | |
| DAY 10 | 0.01 | 0.01 | 0.01 | 0.01 | 0.01 | 0.01 | 0.01 | 0.01 | 0.01 |
| SEVERITY | INCH | INCH | INCH | INCH | INCH | INCH | INCH | INCH | INCH |
| DAY 10 | 3/8 | EDGE | EDGE | 3/4 | 3/4 | 1/8 | 1/8 | 1/8 | EDGE |
| DENSITY | SRF | | | SRF | SRF | SRF | SRF | SRF | |
| DAY 11 | 0.01 | 0.01 | 0.01 | 0.01 | 0.01 | 0.01 | 0.01 | 0.01 | 0.01 |
| SEVERITY | INCH | INCH | INCH | INCH | INCH | INCH | INCH | INCH | INCH |
| DAY 11 | 3/8 | EDGE | EDGE | 3/4 | 3/4 | 1/8 | 1/8 | 1/8 | EDGE |
| DENSITY | SRF | | | SRF | SRF | SRF | SRF | SRF | |
| DAY 14 | 0.01 | 0.01 | 0.01 | 0.01 | 0.01 | 0.01 | 0.01 | 0.01 | 0.01 |
| SEVERITY | INCH | INCH | INCH | INCH | INCH | INCH | INCH | INCH | INCH |
| DAY 14 | 3/8 | 1/8 | 1/8 | 3/4 | 3/4 | 1/8 | 1/8 | 1/8 | EDGE |
| DENSITY | SRF | SRF | SRF | SRF | SRF | SRF | SRF | SRF | |
| DAY 15 | 0.10 | 0.01 | 0.01 | 0.01 | 0.25 | 0.01 | 0.01 | 0.01 | 0.01 |
| SEVERITY | INCH | INCH | INCH | INCH | INCH | INCH | INCH | INCH | INCH |
| DAY 15 | 3/8 | 1/8 | 1/8 | 3/4 | 3/4 | 1/8 | 1/8 | 1/8 | EDGE |
| DENSITY | SRF | SRF | SRF | SRF | SRF | SRF | SRF | SRF | |
| DAY 16 | 0.10 | 0.01 | 0.01 | 0.10 | BRK | 0.01 | 0.01 | 0.01 | 0.01 |
| SEVERITY | INCH | INCH | INCH | INCH | | INCH | INCH | INCH | INCH |
| DAY 16 | 3/4 | 1/8 | 1/8 | 3/4 | BRK | 3/4 | 3/4 | 3/4 | EDGE |
| DENSITY | SRF | SRF | SRF | SRF | | SRF | SRF | SRF | |
| DAY 17 | 0.10 | 0.01 | 0.01 | BRK | BRK | 0.01 | 0.25 | 0.03 | 0.01 |
| SEVERITY | INCH | INCH | INCH | | | INCH | INCH | INCH | INCH |
| DAY 17 | 3/4 | 1/8 | 1/8 | BRK | BRK | 3/4 | 3/4 | 3/4 | EDGE |
| DENSITY | SRF | SRF | SRF | | | SRF | SRF | SRF | |
| DAY 18 | 0.10 | 0.01 | 0.01 | BRK | BRK | 0.01 | 0.25 | BRK | 0.01 |
| SEVERITY | INCH | INCH | INCH | | | INCH | INCH | | INCH |
| DAY 18 | 3/4 | 1/8 | 1/8 | BRK | BRK | 3/4 | 3/4 | BRK | EDGE |
| DENSITY | SRF | SRF | SRF | | | SRF | SRF | | |
| DAY 21 | BRK | 0.10 | 0.25 | BRK | BRK | BRK | BRK | BRK | 0.01 |
| SEVERITY | | INCH | INCH | | | | | | INCH |
| DAY 21 | BRK | 3/4 | 3/4 | BRK | BRK | BRK | BRK | BRK | 3/4 |
| DENSITY | | SRF | SRF | | | | | | SRF |

Compounds A, E, and F shoed antiozonant behavior similar to Zonflax. Compound B failed before Zonflax. Compounds C and D showed behavior similar to Zonflax. Compounds J and N showed significantly better ozone resistance than Zonflax or cyclohexyl PPD. Compounds H, L, and Q showed inferior ozone behavior compared to Zonflax and IPPD.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A compound of Formula I or a salt thereof: 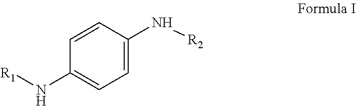
wherein R₁ and R₂ independently comprise linear or branched C3-C20 alkyl; substituted or unsubstituted C3-C20 cycloalkyl, linear or branched C3-C10 alkenyl; fused or bridged C3-C20 cycloalkyl, substituted or unsubstituted C₅-C₁₀ heteroaryl, or substituted or unsubstituted C₅-C₂₀ aryl;
provided that R₂ is not 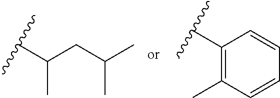
when R₁ is phenyl.

2. The compound or salt of claim 1, wherein R₁ comprises unsubstituted C₅-C₆ aryl or C₅-C₆ aryl substituted with a C₁-C₄ linear or branched alkyl group or a C₅-C₁₀ aryl group.

3. The compound or salt of claim 1 or 2, wherein R₁ and R₂ are different.

4. The compound or salt of any one of claims 1-3, wherein R₁ and R₂ are both aryl.

5. The compound or salt of any one of claims 1-3, wherein R₁ is aryl and R₂ is alkyl or cycloalkyl.

6. The compound or salt of any one of claims 1-3, wherein R₁ and R₂ are both alkyl, both cycloalkyl, or wherein one of R₁ and R₂ is alkyl and one of R₁ and R₂ is cycloalkyl.

7. The compound or salt of claim 1, having the structure:

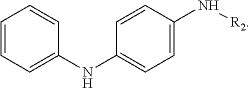

8. The compound or salt of any one of claims 1-7, wherein R₂ is a C3-C20 alkyl group.

9. The compound or salt of any one of claims 1-8, wherein R₂ is selected from

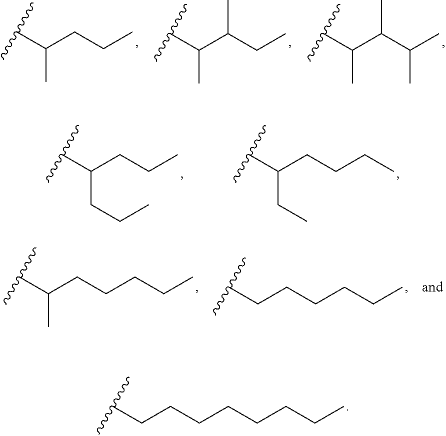

10. The compound or salt of any one of claims 1-7, wherein R₂ is a substituted or unsubstituted C3-C20 cycloalkyl group.

11. The compound or salt of any one of claims 1-7, wherein R₂ is selected from

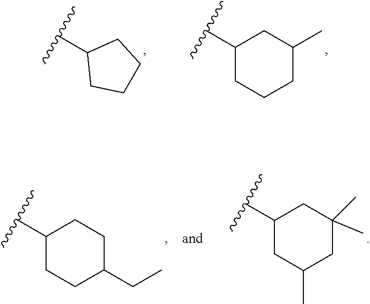

12. The compound or salt of any one of claims 1-7, wherein R₂ is a linear or branched C3-C10 alkenyl group comprising from 1-3 lower alkyl substituents on one double bond.

13. The compound of any one of claims 1-7, wherein R₂ is

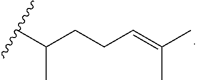 [SMILES: *C(C)CCC=C(C)C]

14. The compound or salt of any one of claims 1-7, wherein R₂ is a fused or bridged C3-C20 cycloalkyl group.

15. The compound or salt of any one of claims 1-7, wherein R₂ is a fused or bridged cycloalkyl group selected from adamantyl, norbornyl, or decalyl.

16. The compound or salt of any one of claims 1-7, wherein R₂ is

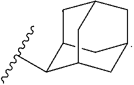 [SMILES: *C1C2CC3CC(C2)C(C)C1C3]

17. The compound or salt of any one of claims 1-16, wherein the compound is selected from

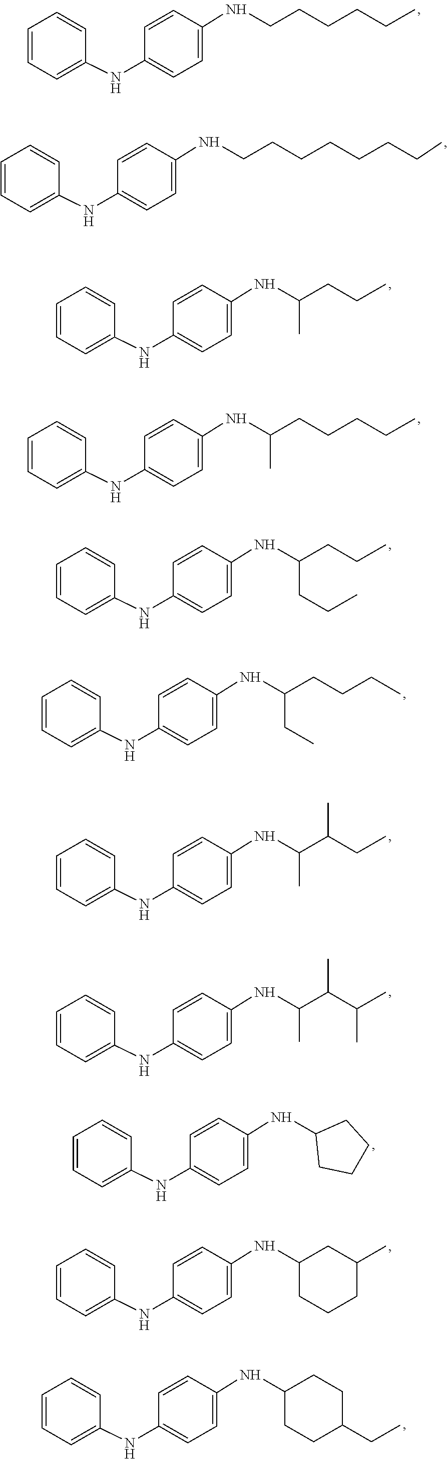

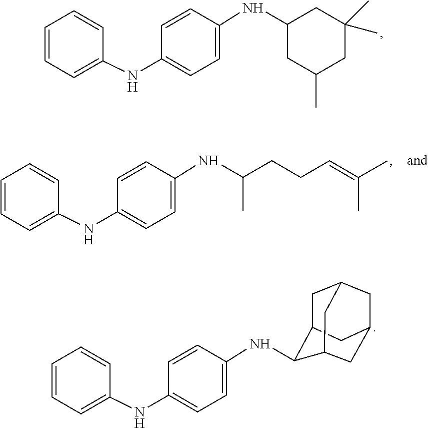 [SMILES: CC1CC(Nc2ccc(Nc3ccccc3)cc2)CC(C)(C)C1 ; CC(C)=CCCC(C)Nc1ccc(Nc2ccccc2)cc1 ; c1ccc(Nc2ccc(NC3C4CC5CC(C4)CC3C5)cc2)cc1]

18. The salt of any one of claims 1-17, wherein the salt has a counter-ion selected from Na⁺, K⁺, Ca²⁺, Mg²⁺, Zn²⁺, or any combination thereof.

19. The compound or salt of any one of claims 1-18, wherein the compound and its degradation products are non-toxic.

20. A method for making the compound or salt of any one of claims 1-19, the method comprising admixing a compound of Formula II 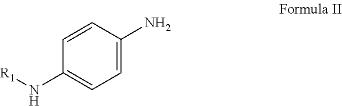
with a carbonyl compound comprising an aldehyde or ketone having Formula III and a reducing agent 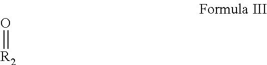
wherein R₁ and R₂ independently comprise linear or branched C3-C20 alkyl; substituted or unsubstituted C3-C20 cycloalkyl, linear or branched C3-C20 alkenyl; fused or bridged C3-C20 cycloalkyl, substituted or unsubstituted C₅-C₁₀ heteroaryl, or substituted or unsubstituted C₅-C₁₀ aryl;
provided that R₂ is not 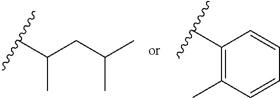
when R₁ is phenyl.

21. The method of claim 20, wherein the compound of Formula I is admixed with the carbonyl compound to produce a first composition, then the first composition is admixed with a reducing agent, optionally in the presence of acetic acid.

22. The method of claim 20 or 21, wherein the method further comprises treating the compound with a base to form the salt.

23. The method of claim 22, wherein the base comprises NaOH, KOH, Ca(OH)₂, Mg(OH)₂, Zn(OH)₂, CaO, K₂CO₃, Na₂CO₃, Na(0), or any combination thereof.

24. The method of any one of claims 20-23, wherein the reducing agent comprises NaBH(OAc)₃, NaBH₄, Na(CN)BH₃, or any combination thereof.

25. The method of any one of claims 20-24, wherein the method is conducted in a polar aprotic solvent.

26. The method of claim 25, wherein the solvent comprises tetrahydrofuran (THF), toluene, xylene, or any combination thereof.

27. A method for making the compound or salt of any one of claims 1-19, the method comprising admixing hydroquinone with R₁—NH₂, R₂—NH₂, and a Lewis acid in a solvent.

28. The method of claim 27, wherein the Lewis acid comprises FeCl₃.

29. The method of claim 27 or 28, wherein the solvent comprises tetrahydrofuran (THF), toluene, xylene, or any combination thereof.

30. A method for making the compound or salt of any one of claims 1-19, the method comprising:
(a) admixing a compound having a structure of Formula II 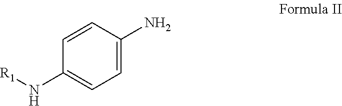
with an aldehyde or ketone having Formula III 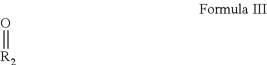
in a solvent to form an intermediate; and
(b) performing catalytic hydrogenation on the intermediate to produce the compound or salt;
wherein R₁ and R₂ independently comprise linear or branched C3-C20 alkyl; substituted or unsubstituted C3-C20 cycloalkyl, linear or branched C3-C20 alkenyl; fused or bridged C3-C20 cycloalkyl, substituted or unsubstituted C₅-C₁₀ heteroaryl, or substituted or unsubstituted C₅-C₁₀ aryl;
provided that R₂ is not 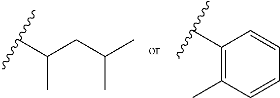
when R₁ is phenyl.

31. The method of claim 30, wherein the solvent comprises tetrahydrofuran (THF), toluene, xylene, or any combination thereof.

32. The method of claim 30 or 31, wherein catalytic hydrogenation is performed using platinum dioxide, cobalt boride, Crabtree's catalyst, HRh[P(C₆H₅)₃]₄, Lindlar catalyst, nickel boride, palladium black, palladium on carbon, Raney nickel, Rh—Pt oxide, Urushibara nickel, Wilkinson's catalyst, or any combination thereof.

33. A rubber composition comprising one or more rubber polymers and the compound or salt of any one of claims 1-19.

34. The rubber composition of claim 33, wherein the one or more rubber polymers comprise natural rubber, polybutadiene, styrene-butadiene rubber, nitrile rubber, polyisoprene, chloroprene, ethylene-butadiene, hydrogenated butadiene, hydrogenated styrene-butadiene, or any combination thereof.

35. The rubber composition of any one of claim 33 or 34, wherein the compound has at least one antiozonant property, at least one antioxidant property, or any combination thereof.

36. The rubber composition of claim 35, wherein the antiozonant property, the antioxidant property, or both comprise prevention of cracking of the one or more rubber polymers, slowing of cracking of the one or more rubber polymers, prevention of a color change of the one or more rubber polymers, preservation of flexibility of the one or more rubber polymers, or any combination thereof.

37. An article comprising the rubber composition of any one of claims 33-36.

38. The article of claim 37, wherein the article is a tire.

39. The article of claim 37 or 38, wherein the compound migrates from an inner surface to an outer surface of the article over time.

40. The article of claim 39, wherein a rate of migration of the compound from the inner surface to the outer surface of the article is within 10% of a rate of migration for a compound selected from

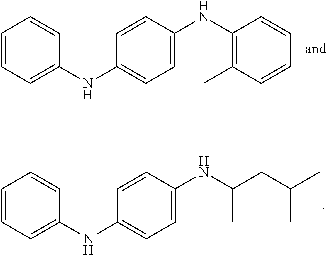 [SMILES: Cc1ccccc1Nc1ccc(Nc2ccccc2)cc1 ; CC(C)CC(C)Nc1ccc(Nc2ccccc2)cc1]

41. A method for enhancing at least one antiozonant property, at least one antioxidant property, or any combination thereof of a rubber composition, the method comprising incorporating the compound or salt of any one of claims 1-19 in a rubber composition.

42. The composition of matter of claim 1-16, wherein the compound is a blend of two or more compounds of Formula I.

43. The blend of compounds or salts of claim 42 having Formula I comprised of:
(a) R₁ is alkyl and R₂ is alkyl, wherein R1 and R2 independently comprise linear or branched C3-C20 alkyl; substituted or unsubstituted C3-C20 cycloalkyl, linear or branched C3-C10 alkenyl; fused or bridged C3-C20 cycloalkyl;
(b) R₁ is alkyl and R₂ is aryl, wherein R1 comprises linear or branched C3-C20 alkyl; substituted or unsubstituted C3-C20 cycloalkyl, linear or branched C3-C10 alkenyl; fused or bridged C3-C20 cycloalkyl, R2 comprises of substituted or unsubstituted C5-C10 heteroaryl, or substituted or unsubstituted C5-C20 aryl;
(c) R₁ is aryl and R₂ is aryl, wherein R1 and R2 independently comprise substituted or unsubstituted C5-C10 heteroaryl, or substituted or unsubstituted C5-C20 aryl;

44. The rubber composition of claim 1-16, wherein the compound is a blend of two or more compounds of Formula I comprised of:
(a) R₁ is alkyl and R₂ is alkyl, wherein R1 and R2 independently comprise linear or branched C3-C20 alkyl; substituted or unsubstituted C3-C20 cycloalkyl, linear or branched C3-C10 alkenyl; fused or bridged C3-C20 cycloalkyl;
(b) R₁ is alkyl and R₂ is aryl, wherein R1 comprises linear or branched C3-C20 alkyl; substituted or unsubstituted C3-C20 cycloalkyl, linear or branched C3-C10 alkenyl; fused or bridged C3-C20 cycloalkyl, R2 comprises of substituted or unsubstituted C5-C10 heteroaryl, or substituted or unsubstituted C5-C20 aryl;
(c) R₁ is aryl and R₂ is aryl, wherein R1 and R2 independently comprise substituted or unsubstituted C5-C10 heteroaryl, or substituted or unsubstituted C5-C20 aryl;
